(19) United States
(12) Patent Application Publication
(10) Pub. No.: US 2025/0226059 A1
(43) Pub. Date: Jul. 10, 2025

(54) VISUALIZING INTRA-CELL CO-EXPRESSION OF CELLULAR CONSTITUENTS USING DIMENSIONALITY REDUCTION THAT IS WEIGHTED BY CELL TYPE

(71) Applicant: BioLegend, Inc., San Diego, CA (US)

(72) Inventor: Santosh Putta, Foster City, CA (US)

(21) Appl. No.: 19/015,300

(22) Filed: Jan. 9, 2025

Related U.S. Application Data
(60) Provisional application No. 63/619,659, filed on Jan. 10, 2024.

(51) Int. Cl. G16B 25/10; G06T 11/00; G16H 30/40

(52) U.S. Cl. CPC G16B 25/10; G06T 11/001; G16H 30/40; G06T 2210/41

(57) ABSTRACT

A facility for analyzing a sample of cells is described. The facility accesses an emphasis weight specifying a degree to which cell type is to be emphasized relative to cell constituent expression levels in determining visualization coordinates for each cell of the sample. The facility generates a cell matrix comprising a grid of values in which each row represents a cell of the sample, in which a first group of the columns correspond to constituent expression levels detected for the cell, and a second group of the columns correspond to a representation established for the cell's cell type, the values in the first group of the columns being weighted against the values in the second group of the columns in accordance with the accessed emphasis weight. The facility performs dimensionality reduction on the rows of the generated cell matrix to obtain visualization coordinates for each cell of the sample.

### RELATED APPLICATIONS

This application claims the benefit of U.S. Provisional Patent Application No. 63/619,659, entitled VISUALIZING INTRA-CELL CO-EXPRESSION OF CELLULAR CONSTITUENTS USING DIMENSIONALITY REDUCTION THAT IS WEIGHTED BY CELL TYPE, filed on Jan. 10, 2024, which is hereby incorporated by reference in its entirety.

In cases in which a document incorporated by reference conflicts with the present application, the present application controls.

### BACKGROUND

Single-cell analysis techniques enable measuring the expression level of different constituents within individual cells, including constituents such as proteins and RNA transcripts. For example, flow cytometry instruments pass single cells through the path of a laser, and interrogate them with various visible and fluorescent light sources that allow assessment of protein composition; mass cytometry instruments apply heavy metal ion tags as labels in place of fluorochromes, and read them using time-of-flight spectrometry; and Cellular Indexing of Transcriptomes and Epitopes by Sequencing (“CITE-Seq”) instruments use DNA-barcoded antibodies to detect proteins.

A common use of these single-cell analysis techniques involves collecting a sample of cells from a particular subject; using an instrument to apply one of the analysis techniques to each cell to obtain an expression level for each of one or more constituents; and outputting a table identifying the measured expression level of each constituent in each cell.

### BRIEF DESCRIPTION OF THE DRAWINGS

FIG. 1 is a block diagram showing some of the components typically incorporated in at least some of the computer systems and other devices on which the facility operates.

FIG. 2 is a flow diagram showing a process performed by the facility in some embodiments in order to generate a visualization of intracell co-expression of cellular constituents.

FIG. 3 is a table diagram showing sample contents of a cell constituent expression level table used by the facility in some embodiments in order to store expression levels detected by a single-cell analysis instrument of a number of cellular constituents in each of a number of individual cells of a cell sample.

FIG. 4 is a hierarchy diagram showing a sample hierarchy used by the facility in some embodiments.

FIG. 5 is a graph diagram showing a sample acyclic hierarchy graph generated by the facility in some embodiments.

FIG. 6 shows sample contents of a cell type adjacency matrix table used by the facility in some embodiments to identify the cell types whose nodes are directly connected in the graph.

FIG. 7 is a table diagram showing sample contents of a cell type frequency table used by the facility in some embodiments to store the frequency in the sample of each assigned cell type-that is, the percentage of cells of the cell sample that were assigned each of the assigned cell types.

FIG. 8 is a table diagram showing sample contents of a weighted cell type adjacency matrix table used by the facility in some embodiments to store weights assigned to edges that directly connect the pairs of nodes in the graph.

FIG. 9 is a table diagram showing sample contents of a weighted cell type distance table used by the facility in some embodiments to store weighted distances between pairs of nodes of the graph representing corresponding pairs of cell types.

FIG. 10 is a table diagram showing sample contents of a normalized weighted cell type distance table used by the facility in some embodiments to store variance-normalized weighted cell type distances.

FIG. 11 is a table diagram showing sample contents of a cell type low-dimensional embedding table used by the facility in some embodiments to store the low-dimensional embeddings of cell type representations created by the facility in act 208.

FIG. 12 is a visualization diagram showing a visualization generated by the facility for the example cell sample using an emphasis weight value of 0.

FIG. 13 is a visualization diagram showing a visualization generated by the facility for the example cell sample using an emphasis weight value of 0.

FIG. 14 is a visualization diagram showing a visualization generated by the facility for the example cell sample using an emphasis weight value of 0.5.

FIG. 15 is a visualization diagram showing a visualization generated by the facility for the example cell sample using an emphasis weight value of 0.75.

FIG. 16 is a visualization diagram showing a visualization generated by the facility for the example cell sample using an emphasis weight value of 0.99.

FIG. 17 is a flow diagram showing a process performed by the facility in some embodiments to generate an alternate visualization in which cell visual representation color represents the expression level of a particular constituent.

FIG. 18 is a visualization diagram showing a visualization generated in accordance with the process shown in FIG. 17.

### DETAILED DESCRIPTION

The inventors have recognized limitations of conventional approaches to single-cell analysis. In particular, they have determined that being able to visualize in two or more dimensions intra-cell co-expression levels in a sample would have significant value. In particular, they recognize that this would provide an improved ability to understand disease biology, perform disease diagnosis, and understand the mechanism of action of drug candidates, as a few examples.

With the help of modern single cell technologies like spectral flow cytometry or CITESeq, it is now possible to measure 10s to 100s of proteins and 1000s of RNA transcripts in 1000s to millions of individual cells. Whether studying cells from healthy subjects or disease states or animal models, there is typically a body of knowledge relating to cell lineage and function that is typically first applied to new data. It is therefore a frequent practice to identify various cell components like T Cells, B Cells, and additional sub-sets using protein or RNA expression; for instance, it is well established that T Cells are lymphocytes that express the protein CD3. Gating is a commonly-used method by which domain experts will assign individual cells, often in a hierarchical manner, to various cell types. Often these cell types are well understood either from a lineage or functional perspective. Deriving additional insights from single cell data is aided by this cell categorization; i.e., new discoveries are best understood in the context of known cell types and sub-types. Cell typing often involves a handful of the dimensions (usually protein markers) that are measured on each cell. The expression patterns for these markers are well understood in the literature; for instance, T Cells are known to express CD3 while B Cells are known to express CD19. In addition, the lineage of these cell types (e.g., T and B Cells are a subset of lymphocytes) is also well established. Gating therefore takes advantage of this knowledge of lineage and expression patterns, typically visualized using one of two-dimensional plots, and to then identify cell types.

For example, typical gating plots may show CD14 and CD45 expression used to identify Monocytes and Lymphocytes, followed by CD3 and CD19 expression in Lymphocytes used to identify T Cells and B Cells, followed by CD4 and CD8 expression in T Cells used to identify CD4+ T Cells and CD8+ T Cells. In a plot, each point represents a cell, the color represents the density of cell in a particular region of the plot—red indicates many cells with data values close to each other while blue indicates a sparse distribution.

Alternate methods like clustering are also employed to identify cell types, often in a more exploratory setting when the cell lineage is not well understood or to further sub-divide a cell type following gating.

The goal for defining cell types is to create the context for additional data analysis. The number of markers (typically protein markers) used for defining cell types is typically a handful (10-20 markers). On the other hand, it is now possible to measure many (100s to 1000s) more protein and RNA markers. Analyzing these additional markers in the context of the cell types and disease or other information relating to the biospecimen is highly valuable. As such, it is frequent practice to quantify protein, RNA, or other markers, in particular those markers that have not been used to define cell types, within these cell types for further analysis. Visual analysis of the single cell data, to supplement the quantification, is also highly desirable to fully understand the heterogeneity of marker expression (and co-expression) across the cell types. Given the high-dimensional nature of the data, visualization is often challenging. Dimensionality reduction methods like t-SNE and UMAP are often applied to this high-dimensional single cell data to visualize various cellular components and the expression of protein or RNA or other markers in these components. Broadly speaking, these non-linear methods attempt to learn the high-dimensional manifold of the data and provide a lower (often two) dimensional approximation. They attempt to preserve the relationships (distances) between cells that are close to each other in the high-dimensional space; i.e., the neighborhood, when reducing to lower dimensional space at the expense of distorting distances between cells that are far from each other.

While dimensionality reduction provides a convenient way to visualize the data in lower dimensions, it is conventionally computed independently from defining cell types. As a result, it is not guaranteed that in the lower dimensional space biologically meaningful cell types will be visually separated to easily facilitate other marker analysis within that context. The inventors have recognized that cell types often overlap in lower dimensional space, making it challenging to understand differential expression of markers within and across the cell types.

The inventors have determined that this overlap or smudging of cell types in the projection space can happen for two reasons. First, t-SNE or similar algorithms are typically applied to dimensions (protein and RNA expression) beyond those involved in cell typing. This is often desirable, because a goal for visual exploration is to understand if there is protein/RNA expression that is responsible for sub-clusters of cells beyond cell typing. In other words, there is a natural competition between the lineage markers used for cell type definition and the rest that may have similar expression across cell types. Leaving the non-lineage markers out of dimensional reduction may improve the separation between cell types but will provide limited insight into any sub-clusters of cells due to expression of other markers. A second reason for the overlap is because the cell lineage markers can have substantial variance across samples either due to subject-to-subject variation or due to ex vivo treatment of cells. For example, ex vivo activation of T Cells will change several T Cell markers, but still provide sufficient resolution to define T Cells and relevant subsets via gating. The change in lineage markers from sample to sample will mean that in the projection space, the same cell types can appear in different areas for different samples, making visual analysis more difficult. More importantly, the user is already aware that lineage markers vary between samples, so the projections highlighting this difference at the expense of diluting the patterns in other markers is typically not beneficial during analysis.

Based on this recognition, the inventors have conceived and reduced to practice a software and/or hardware facility for visualizing intra-cell co-expression of cellular constituents such as proteins and RNA transcripts using dimensionality reduction that is weighted by cell type (“the facility”).

Conventional dimensionality reduction methods assess if two points (e.g., cells) are similar or dissimilar. This assessment of similarity is first done in a high-dimensional (“original”) space. The method then seeks to preserve this similarity and dissimilarity between cells to the best extent possible in a lower-dimensional (projection) space. For example, the t-SNE dimensionality reduction methods computes Euclidean distance in the high dimensional space and converts it to a probabilistic measure of similarity to generate a probability distribution. Then via a minimization procedure, t-SNE attempts to preserve the probability distribution of the high dimensional space in the low dimensional space. t-SNE is further described by Visualizing Data using t-SNE, Laurens van der Maaten, Geoffrey Hinton, Journal of Machine Learning Research 9 (2008) 2579-2605, which is hereby incorporated by reference in its entirety.

While UMAP is methodologically different from t-SNE, it also relies on which cells are close (similar) to each other and which are not. UMAP computes a Manhattan distance to determine points/cells that are closer to each and connects them with a higher probability to create a graph representation of the probability data. UMAP then attempts to preserve the graph's structure in the low-dimensional space. Both the distance metrics (e.g., Manhattan instead of Euclidean) and other tunable parameters that define the size of the neighborhood can be changed to obtain optimal projections for diverse types of datasets. UMAP is described further by UMAP: Uniform Manifold Approximation and Projection, Leland McInnes, John Healy, Nathaniel Saul, Lukas, Großberger Journal of Open Source Software, 3(29), 861, https://doi.org/10.21105/joss.00861, which is hereby incorporated by reference in its entirety.

Relative to conventional dimensionality reduction methods, the facility adjusts the points/cells that are similar to each other in such a way that those cells belonging to the same cell type have a higher probability of being similar to each other.

In some embodiments, the facility subjects single-cell analysis instrument output data for a single subject's cell sample, or “well,” to a process—such as gating or clustering—that attributes a cell type to each cell in the sample based upon their co-expression levels of combinations of constituents that are characteristic of different cell types. As one example, in some embodiments, the facility operates to assess co-expression of proteins in tumor infiltrating cells extracted from lung cancer patients. In some embodiments, samples from different subjects are pooled.

For each of the cell types attributed to cells of a particular sample, the facility establishes a low-dimensionality representation of the cell type—such as a two-dimensional representation—such that similar attributed cell types are nearer to one another than dissimilar attributed cell types. In some embodiments, this involves determining a hierarchy relating the attributed cell types; generating an acyclic graph representing the hierarchy; determining for each pair of attributed cell types a distance in the graph, which in some cases is adjusted to reflect the frequency of the cell types of the pair within the sample; determining a high-dimensionality representation of each cell type based on its graph distances from other attributed cell types; and applying a dimensionality reduction technique to these high-dimensionality representations of each cell type to obtain the low-dimensionality representation as an embedding.

The facility determines an emphasis weight specifying the degree to which cell type is to be emphasized relative to cell constituent expression levels in the visualization it produces, such as by receiving the emphasis weight as user input. The facility generates a cell matrix, in which each row of the cell matrix represents a cell of the sample, and the cell matrix includes two groups of columns: (a) variance-normalized versions of the expression levels detected in the cell for each cellular constituent, and (b) the low-dimensional embedding of the cell's cell type. The numbers of columns in the first group and the second group vary based on the embodiment. In the cell matrix, the values in columns in the first group are weighted against the values in the columns of the second group using the emphasis weight. The facility subjects each row of the cell matrix to a dimensionality reduction technique to obtain visualization coordinates that are used to plot a visual representation of the cell in a low-dimensional visualization space, such as a two-dimensional visualization space.

The inventor has used a neighborhood purity measure reflecting the degree of colocation of cells of the same cell type as a basis for evaluating the visualizations produced by the facility. On a per-cell-type basis, the inventor has observed significant increases in neighborhood purity in the visualizations produced by the facility, as contrasted with conventional visualizations that do not consider cell type, cell type lineage, or cell type hierarchy.

In some embodiments, the facility identifies for a single cell type, one or more constituents that weren't used in identifying cells of that cell type, but whose expression levels steer particular cells to different sub-clusters within the cell type. It does so by choosing a constituent, and generating a new visualization in which the color of the visual indication for each cell is based on the expression level of the chosen constituent.

By operating in some or all of the ways described herein, the facility enables users to easily visualize constituent co-expression in a cell sample, including identifying, for a single cell type, one or more constituents that weren't used in identifying cells of that cell type, but whose expression levels steer particular cells to different sub-clusters within the cell type.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by generating dimensionality-reduced visualizations that are more useful than conventional approaches, the facility avoids the processor cycles and memory occupancy that would otherwise be needed to keep repeating the conventional process in pursuit of better and more useful visualizations.

Further, for at least some of the domains and scenarios discussed herein, the processes described herein as being performed automatically by a computing system cannot practically be performed in the human mind, for reasons that include that the starting data, intermediate state(s), and ending data are too voluminous and/or poorly organized for human access and processing, and/or are a form not perceivable and/or expressible by the human mind; the involved data manipulation operations and/or subprocesses are too complex, and/or too different from typical human mental operations; required response times are too short to be satisfied by human performance; etc.

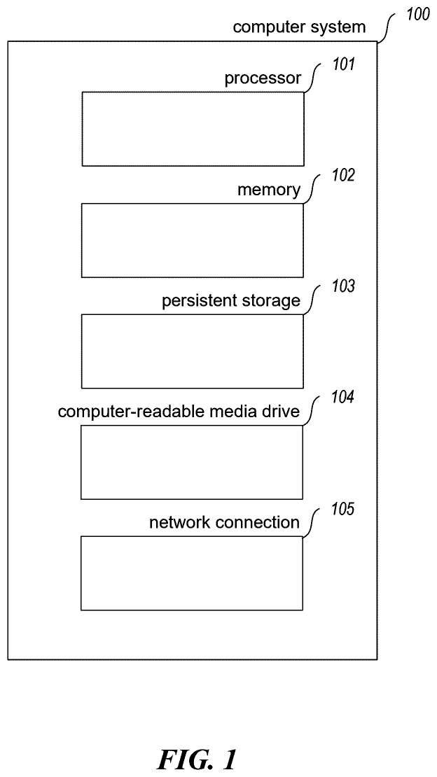

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102—such as RAM, SDRAM, ROM, PROM, etc.—for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. None of the components shown in FIG. 1 and discussed above constitutes a data signal per se. While computer systems configured as described above are typically used to support the operation of the facility, those skilled in the art will appreciate that the facility may be implemented using devices of various types and configurations, and having various components.

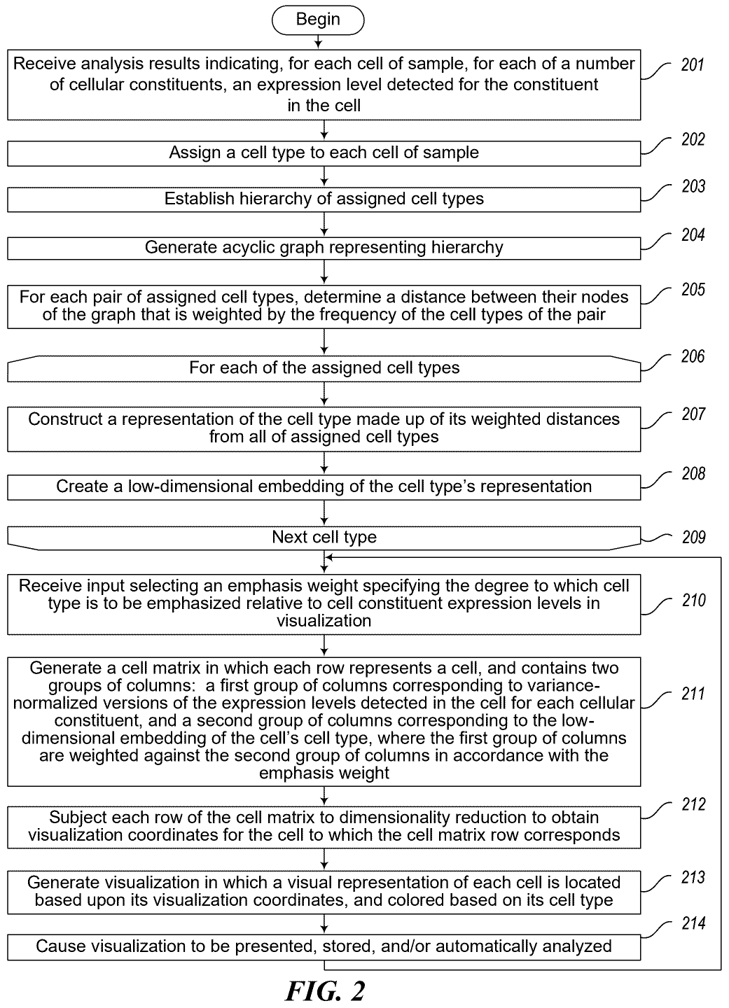

FIG. 2 is a flow diagram showing a process performed by the facility in some embodiments in order to generate a visualization of intracell co-expression of cellular constituents. In act 201, the facility receives analysis results directly or indirectly from a single-cell analysis instrument. The analysis results indicate for each cell of a sample of cells processed by the analysis instruments—such as a sample obtained from a single human or other animal patient or other subject—an expression level detected for each of a number of cellular constituents in that cell. These analysis results may be stored, for example, in a data table.

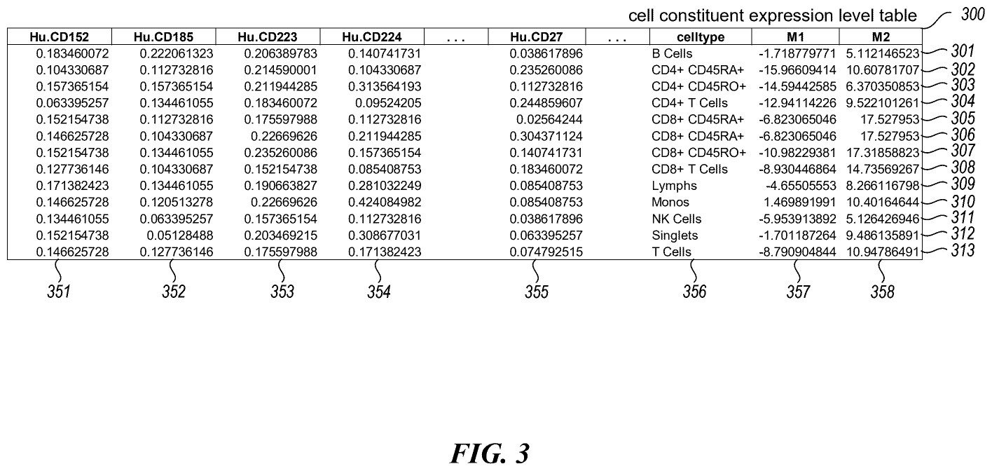

FIG. 3 is a table diagram showing sample contents of a cell constituent expression level table used by the facility in some embodiments to store expression levels detected by a single-cell analysis instrument of a number of cellular constituents in each of a number of individual cells of a cell sample. The cell constituent expression level table 300 is made up of rows, such as rows 301-313, each corresponding to a different cell of the sample. Each row is divided into a number of columns. The columns include columns such as columns 351-355, each corresponding to a different cellular constituent, and containing a value for the expression level for that cellular constituent in the cell to which each particular row corresponds. For example, column 351 has contents indicating expression levels in different cells of the cellular constituent Hu.CD152. The value at the intersection of column 351 with row 301—0.183460072—indicates the expression level of this cellular constituent in the particular cell to which row 301 corresponds. Additional columns in the cell constituent expression level table 300 are discussed herein.

While FIG. 3 and each of the table diagrams discussed herein show a table whose contents and organization are designed to make them more comprehensible by a human reader, those skilled in the art will appreciate that actual data structures used by the facility to store this information may differ from the table shown, in that they, for example, may be organized in a different manner; may contain more or less information than shown; may be compressed, encrypted, and/or indexed; may contain a much larger number of rows than shown, etc. Additionally, in some embodiments, rather than storing the data shown in the table diagrams in tables, the facility stores it in semi-structured or unstructured data stores, such as JSON objects.

Returning to FIG. 2, in act 202 the facility assigns a cell type to each cell of the sample. For example, in various embodiments, the facility uses cell type identification techniques such as gating and clustering. Clustering techniques are described, for example, in Leiden: From Louvain to Leiden: guaranteeing well-connected communities, V. A. Traag, L. Waltman & N. J. van Eck, available at arxiv.org/abs/1810.08473; and PARC: Ultrafast and accurate clustering of phenotypic data of millions of single cells, Shobana V Stassen, Dickson M D Siu, Kelvin C M Lee, Joshua W K Ho, Hayden K H So, Kevin K Tsia; Bioinformatics, Volume 36, Issue 9, May 2020, Pages 2778-2786, each of which are hereby incorporated by reference in its entirety.

Returning to FIG. 3, the cell constituent expression level table also includes a cell type column 356 in which the facility records the cell type identified for each cell in act 202. For example, the value at the intersection of column 356 with row 301 indicates that the cell to which row 301 corresponds has been assigned the cell type “B Cells.”

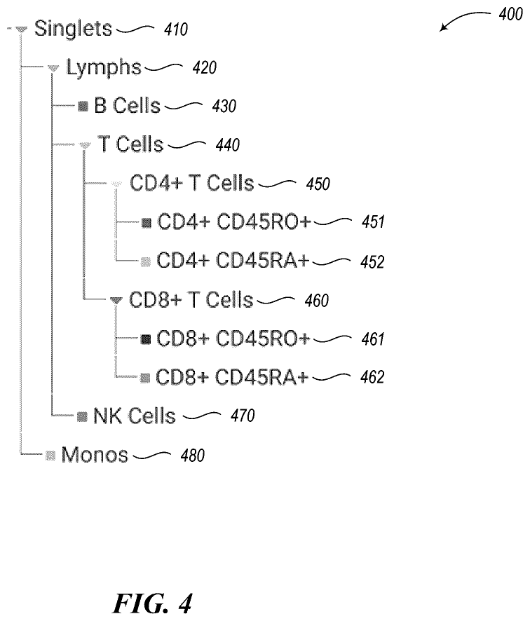

Returning to FIG. 2, in act 203, the facility establishes a hierarchy organizing all of the cell types assigned by the facility across the cell sample in act 202. In establishing this hierarchy, the facility seeks to establish a structure in which the cell types that are most similar in one or more respects are nearby one another in the hierarchy. In some embodiments, the hierarchy established by the facility reflects the understood evolutionary lineage of particular cells types, such that a first cell type is established as a child of a second type in the hierarchy if cells of the first type are thought to evolve from cells of the second type. For example, in some embodiments, the facility establishes Lymphs 420 node as a child of Singlets 410 node in the hierarchy 400 shown in FIG. 4 discussed below reflects an understanding that lymph cells evolved from singlets. In some embodiments, step 203 involves automatically filtering a preexisting hierarchy of all of the cell types it is possible to assign in act 202 to include only the cell types that are actually assigned in step 202 for the present cell sample.

In some embodiments, the facility enables a user of the facility to determine the hierarchy used by the facility. In some such embodiments, for example, the facility provides a user interface showing a proposed hierarchy, in which the user can manipulate the hierarchy by moving the nodes representing different cell types to new positions within the hierarchy.

FIG. 4 is a hierarchy diagram showing a sample hierarchy used by the facility in some embodiments. In the hierarchy 400, child cell types are indented below their parent cell types. For example, in hierarchy 400, CD4+ CD45RA+ cell type 451 is a child of CD4+ T Cells cell type 450, which is in turn a child of T Cells cell type 440, which is in turn a child of Lymphs cell type 420, which is in turn a child of Singlets cell type 410. The cell types shown in the hierarchy are those assigned to the cells of the sample whose cells are shown in the cell constituent expression level 300 shown in FIG. 3.

In various embodiments, the facility uses a variety of hierarchies of cell types other than shown in FIG. 4. Table 1 below shows a hierarchy of cell types used by the facility in some embodiments in connection with flow cytometry cell gating.

TABLE 1

```
Ungated
  Singlets
    Live
      CD45+
        Lymphocytes
          CD3− TCRgd−
            Lin−
              Lin− DR+
                CD11c+ DCs
                  CD11c+ CD16− DCs
                    CD141+ DCs
                    CD1c+ DCs
                  CD11c+ CD16+ DCs
                CD123+ pDCs
            CD3− TCRgd− CD19− CD20−
              CD14− CD123−
                CD14− CD123− DR− CD16−
                  CD14− CD123− DR− CD16− CD4− CD8−
                    ILCs
            CD19+ CD20+
              Naive B Cells
              Memory B Cells
                IgD− Memory
                Plasmablasts
              Marginal Zone-like
          CD3+ TCRgd−
            CD3+ CD56−
              CD8+ T Cells
                CD8+ CCR7− CD45RA−
                  Terminal Effector CD8+
                  Early-like Effector CD8+
                  Early Effector CD8+
                  Intermediate Effector CD8+
                CD8+ CCR7− CD45RA+
                CD8+ CCR7+ CD45RA+
                CD8+ CCR7+ CD45RA−
              CD4+ T Cells
                Tregs
                CD4+ CCR7− CD45RA−
                  Terminal Effector CD4+
                  Early-like Effector CD4+
                  Early Effector CD4+
                  Intermediate Effector CD4+
                CD4+ CCR7− CD45RA+
                CD4+ CCR7+ CD45RA+
                CD4+ CCR7+ CD45RA−
              CD4− CD8−
            CD3+ CD56+
          CD3+ TCRgd+
            TCRgd+ CCR7− CD45RA+
            TCRgd+ CCR7+
            TCRgd+ CCR7− CD45RA−−
        Monocytes
          Non-classical
          Classical
          Intermediate
      CD45 into CD123+
        CD123+ Basophils
```

Table 2 below shows a cell type hierarchy used by the facility in some embodiments in connection with CITESeq gating.

TABLE 2

```
Singlets
  Lymphocyte
    CD3− TCRgd−
      Lin−
        Lin− DR+
          CD11c+ DCs
            CD11c+ CD16+ DCs
            CD11c+ CD16− DCs
              CD1c+ DCs
              CD141+ DCs
          CD123+ pDCs
      CD3− TCRgd− CD19− CD20−
        CD14− CD123−
          CD14− CD123− DR− CD16−
            CD14− CD123− DR− CD16− CD4− CD8−
              ILCs
      CD19+ CD20+
        Naive B Cells
        Memory B Cells
          Plasmablasts
          IgD− Memory
        Marginal Zone-like
    CD3+ TCRgd−
      CD3+ CD56−
        CD8+ T Cells
          CD8+ CD62L+ CD45RA+
          CD8+ CD62L+ CD45RA−
          CD8+ CD62L− CD45RA−
            Terminal Effector CD8+
            Early-like Effector CD8+
            Intermediate Effector CD8+
            Early Effector CD8+
        CD4+ T Cells
          Tregs
          CD4+ CD62L− CD45RA−
            Terminal Effector CD4+
            Early-like Effector CD4+
            Early Effector CD4+
            Intermediate Effector CD4+
          CD4+ CD62L+ CD45RA−
          CD4+ CD62L+ CD45RA+
        CD4− CD8−
      CD3+ CD56+
    CD3+ TCRgd+
  Monocytes
    Classical
    Non-classical
    Intermediate
```

Returning to FIG. 2, in act 204, the facility generates an acyclic graph representing the hierarchy established in act 203. In some embodiments, this involves creating a node representing each of the cell types that appears in the established hierarchy, and connecting each child node to the node of its parent cell type by an edge.

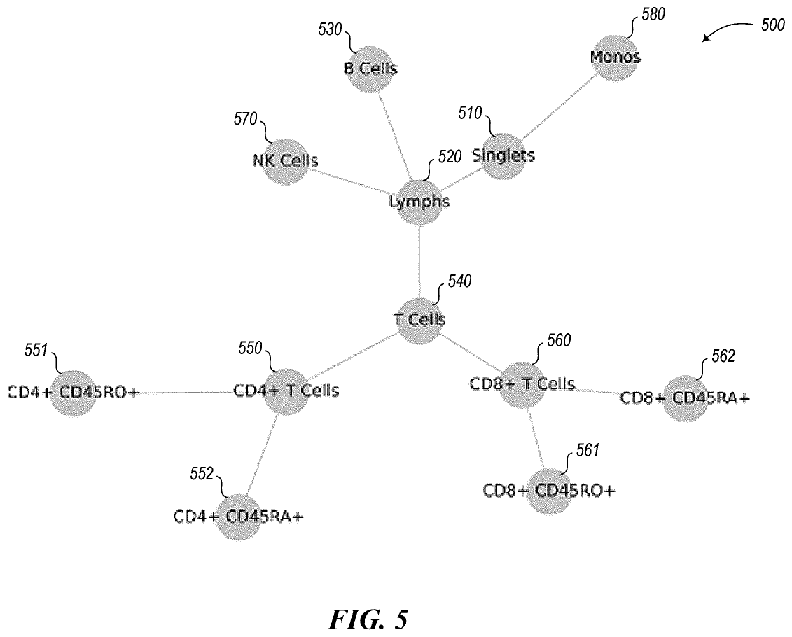

FIG. 5 is a graph diagram showing a sample acyclic hierarchy graph generated by the facility in some embodiments. The graph 500 is made up of nodes connected by edges. Each of the nodes corresponds to one of the cell types from cell type hierarchy shown in FIG. 4. For example, it can be seen, that a Singlets node 510 corresponding to singlets cell type 410 is connected to a Lymphs node 520 corresponding to the Lymphs cell type 420, which is in turn connected to a T Cells node 540 corresponding to the T Cells cell type 440, and so on.

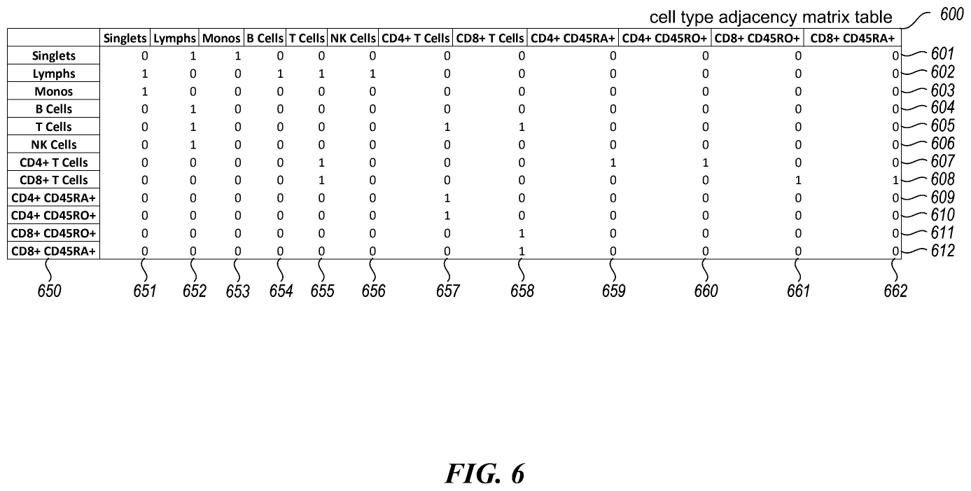

Returning to FIG. 2, in act 205, for each distinct pair of the cell types assigned in act 202, the facility determines a distance between their nodes of the graph that is weighted by the frequency of each of the cell types of the pair among the cells of the cell sample. Details of act 205 are discussed below with respect to FIGS. 6-10.

FIG. 6 shows sample contents of a cell type adjacency matrix table used by the facility in some embodiments to identify the cell types whose nodes are directly connected in the graph. The cell type adjacency matrix table 600 is made up of rows, such as rows 601-612, each corresponding to one of the assigned cell types, shown in column 650. Each row further contains additional columns 651-662 that also correspond to the assigned cell types. At each intersection of a row with a substantive column 651-662, the value indicates whether the pair of cell types represented by the row and the column are directly connected-that is connected by a single edge. For example, the intersection of row 602 with column 651 indicates that the node for the Lymphs cell type is directly connected to the node for the Singlets cell type.

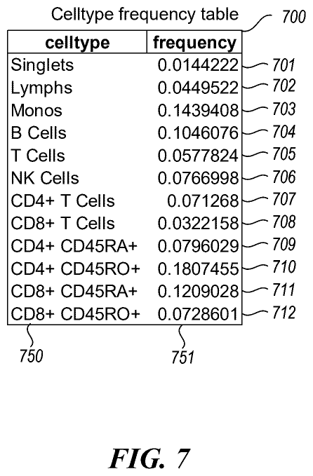

FIG. 7 is a table diagram showing sample contents of a cell type frequency table used by the facility in some embodiments to store the frequency in the sample of each assigned cell type—that is, the percentage of cells of the cell sample that were assigned each of the assigned cell types. For example, row 701 indicates that the frequency with which Singlets cell type was assigned to cells of the sample is 0.0144222—that is, 1.44222 percent of the cells of the sample were assigned the Singlets cell type.

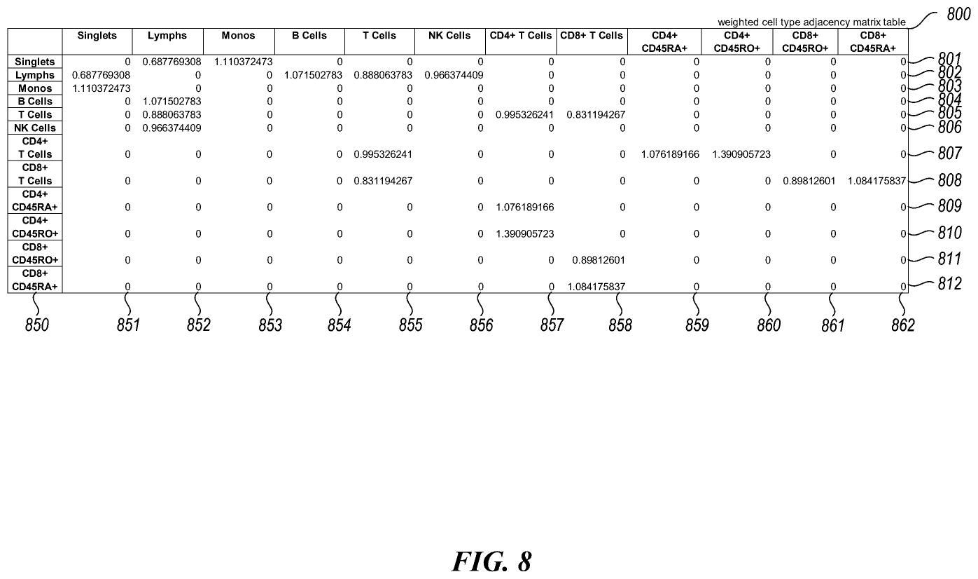

FIG. 8 is a table diagram showing sample contents of a weighted cell type adjacency matrix table used by the facility in some embodiments to store weights assigned to edges that directly connect the pairs of nodes in the graph. The organization of weighted cell type adjacency matrix table 800 corresponds to the organization of cell type adjacency matrix table 600 shown in FIG. 6. The facility produces the weighted cell type adjacency matrix table 800 by replacing each value of 1 in the cell type adjacency matrix table 600 with a weight determined by the formula:

$$B [ i , j ] = sqrt ( Fi + Fj )$$

where Fi and Fj are the frequencies for cell type i and j respectively. For example, the values 0.687769308 at the intersection of column 851 with row 802 indicates that weight for the combination of the Lymphs cells type with the Singlets cell type, obtained by taking the square root of the sum of the frequencies of these cell types, 0.0144222 and 0.0449522 shown in rows 701 and 702 of the cell type frequency table 700 shown in FIG. 7.

The facility then proceeds to use the contents of the weighted cell type adjacency matrix table 800 to construct a weighted cell type distance table that contains a weighted distance in the graph between each distinct pair of cell types, including both those that are connected directly and indirectly in the graph. The facility does this by finding the shortest path between the nodes representing each pair of cell types and summing the weights for each edge that is traversed in that path that are shown in the weighted cell type adjacency matrix table 800.

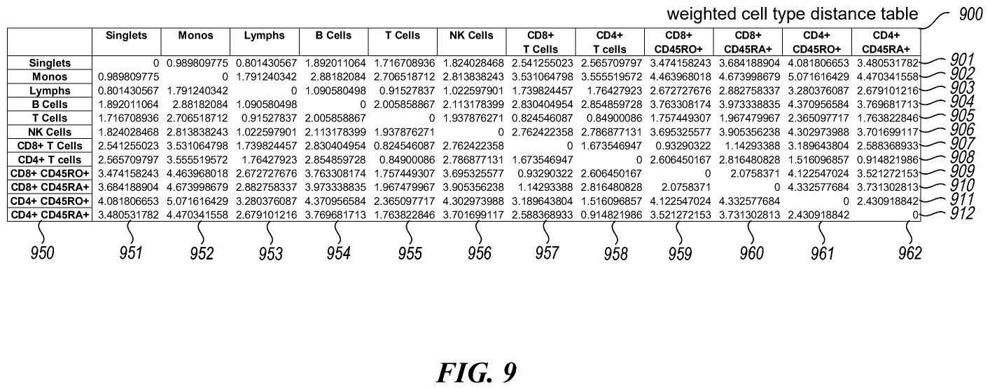

FIG. 9 is a table diagram showing sample contents of a weighted cell type distance table used by the facility in some embodiments to store weighted distances between pairs of nodes of the graph representing corresponding pairs of cell types. The organization of weighted cell type distance table 900 mirrors those of cell type adjacency matrix table 600 and weighted cell type adjacency matrix table 800. The contents of each intersection of a row with a substantive column is determined in the manner discussed above: finding the shortest path between the nodes corresponding to the cell types of the row and column, and summing the weights in weighted cell type adjacency matrix table 800 for the edges in that path. For example, the value 1.716708936 at the intersection of column 955 for T Cells and row 901 for Singlets is obtained by the facility by summing 0.687769308 at the intersection of row 801 for Singlets and 852 for Lymphs with 0.888063783 at the intersection of column 855 for T Cells and row 802 for Lymphs, representing the weights of the edges between the Singlets node 510 of the graph and the T Cells 540—i.e., the edge between Singles node 510 and Lymphs node 520, and the edge between Lymphs node 520 and T Cells node 540. In some embodiments, the facility variance-normalizes the weighted cell type distances shown in weighted cell type distance table 900.

Note that the CTDs, generated from the graph representation of the cell types, are in an arbitrary coordinate system that is unrelated to the BDs. Variance normalization puts the coordinate systems for CTDs and BDs on an even footing. Then weighting for CTDs and BDs described below is directly related to the desired contributions from CTDs and BDs. Variance normalization simply divides BDs by the sum of standard deviation across all BDs. The same is done to normalize CTDs; I.e. divide CTDs by the sum of standard deviates across all CTDs. Alternate forms of normalization or more broadly coordinate transformation for BDs and CTDs to make them compatible with each other are possible. Note that all cells belonging to the same cell type will be set to the same values for the CTDs. Since the number of BDs is typically much larger than that for CTDs and is variable from one data set to another, normalizing BDs and CTDs by the respective total variance puts them on a similar scale prior to combining them to create the augmented data matrix. The adjustable weight w provides a convenient mechanism to change the relative importance of BDs compared to CTDs in the final steps of dimensionality reduction discussed in the next step.

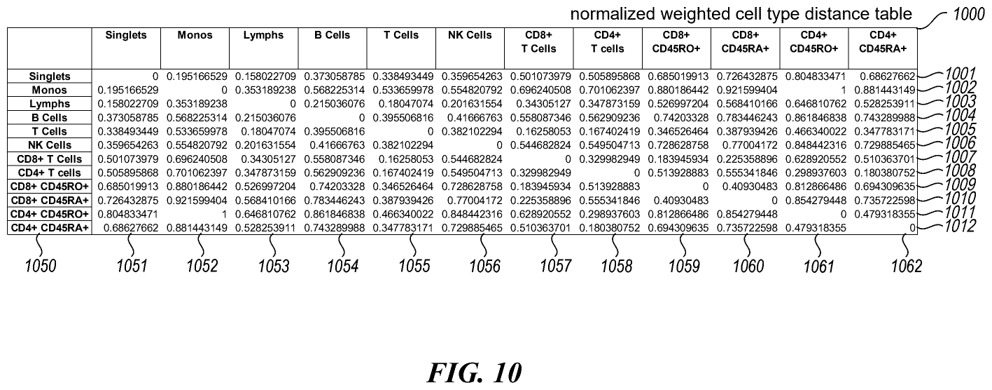

FIG. 10 is a table diagram showing sample contents of a normalized weighted cell type distance table used by the facility in some embodiments to store variance-normalized weighted cell type distances. The organization of normalized weighted cell type distance table 1000 mirrors those of the tables shown in FIGS. 6, 8, and 9 discussed above. The values produced in this table are obtained by the facility in the manner described above.

Returning to FIG. 2, in acts 206-209 the facility loops through each of the cell types assigned in act 202. In act 207, the facility constructs a representation of the cell type that is made up of its weighted distances from all of the cell types assigned in act 202. For example, in some embodiments, this constructed representation is a concatenation of these distances, in an order that is consistent among the constructed representations. In act 208, the facility creates a low-dimensional embedding of the cell type's representation. In act 209, if additional cells types remain to be processed, then the facility continues in act 206 to process the next cell type, else the facility continues in act 210.

In some embodiments, the facility performs the processing shown in acts 206 and 209 using techniques such as t-SNE; multi-dimensional scaling (“MDS”); or graph layout methods such as Graph Drawing by Force-Directed Placement, Fruchterman, Thomas M. J.; Reingold, Edward M. (1991), Software: Practice and Experience, Wiley, 21 (11): 1129-1164, doi: 10.1002/spe.4380211102; An algorithm for drawing general undirected graphs, Kamada, Tomihisa; Kawai, Satoru (1989), Information Processing Letters, Elsevier, 31 (1): 7-15, doi: 10.1016/0020-0190 (89) 90102-6; and Spring Embedders and Force-Directed Graph Drawing Algorithms, Kobourov, Stephen G. (2012), available at arxiv.org/abs/1201.3011, each of which is hereby incorporated by reference in its entirety.

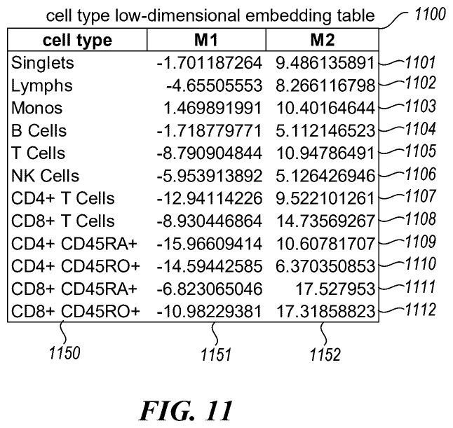

FIG. 11 is a table diagram showing sample contents of a cell type low-dimensional embedding table used by the facility in some embodiments to store the low-dimensional embeddings of cell type representations created by the facility in act 208. Each row of the cell type low-dimensional embedding table 1100 corresponds to a different cell type, shown in column 1150. Each row further contains two values in columns 1151 and 1152 that together make up the embedding—in other words, each embedding is a two-dimensional embedding. For example, row 1101 indicates that the facility has created for the Singlets cell type a low-dimensional embedding of (−1.701187264, 9.486135891). Again, these embeddings are assigned by the facility in such a way as to create distances in two dimensional space between the embeddings of pairs of cell types that reflect the level of similarity of those cell types.

Returning to FIG. 2, in act 210, the facility receives input selecting an emphasis weight specifying the degree to which cell type is to be emphasized relative to cell constituent expression levels in visualization generated by the facility. In some embodiments, this emphasis weight, W, is 0 to emphasize cellular constituents to the total exclusion of cell type, 1 to emphasize cell type to the total exclusion of cellular constituents, or intermediate values representing different mixes of these two considerations.

In act 211, the facility generates a cell matrix in which each row represents a cell, and that row contains two groups of columns, with a first group of the columns corresponding to variance-normalized versions of the expression levels detected in the cell for each cellular constituent, and a second group of columns corresponding to the low-dimensional embedding of the cell's cell type, where the first group of the columns are weighted against the second group of the columns in accordance with, or using, the emphasis weight. In some embodiments, the facility uses the formula below to populate the cell matrix.

$${F}_{N \times ( M + L )} = [ ( 1 - w ) * {B}_{N \times M} \vert W * {C}_{N \times L} ]$$

N: number of cells
M: number of constituent dimensions
L: number of dimensions for cell type embedding, e.g., 2
B_{N×M}: Variance calibrated matrix of protein/RNA data
C_{N×L}: Variance calibrated matrix of cell type embedding
w: Adjustable weight

In some embodiments, the cell matrix generated by the facility in act 211 constitutes a grid of values in which each row represents each of the cells of the sample, in which a first group of the columns correspond to constituent expression levels detected for each of the cells, and a second group of the columns correspond to a representation established for each of the cell's cell type, the values in the first group of the columns being weighted against the values in the second group of the columns in accordance with the emphasis weight.

In some embodiments, the cell matrix generated by the facility in act 211 constitutes a grid of values in which each row represents one of the cells of the sample, in which a first group of the columns correspond to constituent expression levels detected for the cell, and a second group of the columns correspond to a representation established for the cell's cell type, the values in the first group of the columns being weighted against the values in the second group of the columns in accordance with the emphasis weight. The cell constituent expression level Table 300 shown in FIG. 3 is an example of such a cell matrix, in which each of rows 301-313 represents one of the cells of the sample; columns 351-355 are a first group of columns corresponding to constituent expression levels detected for the cell, and columns 357 and 358 are a second group of columns that correspond to a representation established for the cell's cell type.

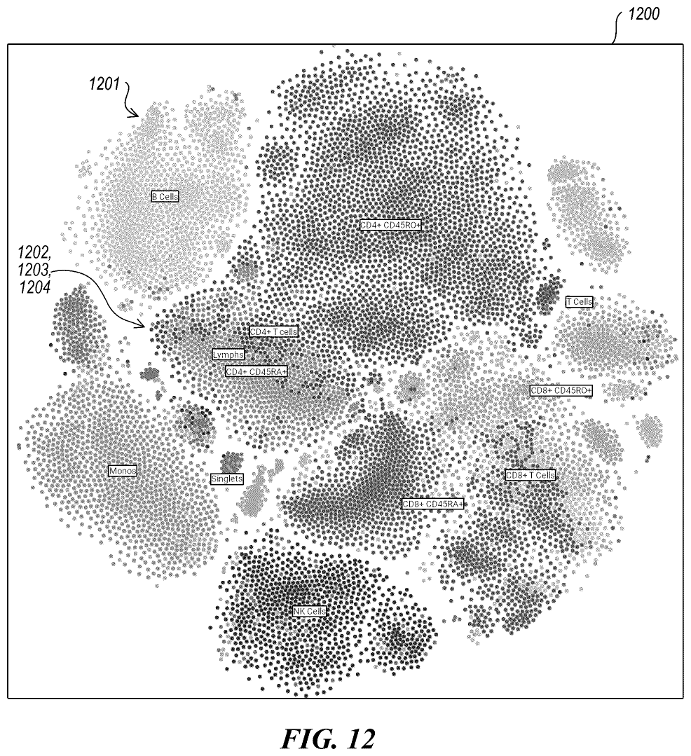

In act 212, the facility subjects each row of the cell matrix to a dimensionality reduction operation to obtain visualization coordinates for the cell to which the cell matrix row corresponds. For example, to generate a two-dimensional visualization, the facility performs dimensionality reduction to produce a two-dimensional representation of reach row. In various embodiments, the facility employs a variety of dimensionality reduction techniques, including t-SNE or UMAP described above. In act 213, the facility generates a visualization for the cell sample in which a visual representation of each cell is located based upon the visualization coordinates obtained for it in act 202, and the visual representation of the cell is colored based upon the cell type assigned to the cell. Various visualizations generated by the facility in act 213 for the cell sample represented in the cell constituent expression level table 300 are shown in FIGS. 12-16, discussed below. In act 214, the facility causes the visualization generated in act 213 to be presented, stored, and/or automatically analyzed. After act 214, the facility continues in act 210 to receive new input selecting a different emphasis weight, and repeats the process of generating a visualization using this new emphasis weight.

Those skilled in the art will appreciate that the acts shown in FIG. 2 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

FIGS. 12-16 show visualizations generated by the facility for the cell sample shown in cell constituent expression level table 300. FIG. 12 is a visualization diagram showing a visualization generated by the facility for the example cell sample using an emphasis weight value of 0. In the visualization 1200, each circle represents a cell of the cell sample, and its color corresponds to the cell type assigned to it. Also, rectangular textual labels are placed at a point near the center of the cell locations for each cell type. For example, the label “B Cells” is near the center of the bluish cluster of cells 1201 representing the cells of the sample assigned the cell type B Cells. It can be seen in this visualization, where cellular constituents are emphasized to the complete exclusion of cell types as a result of selecting W=0, that many cell types are intermingled. For example, the region having reference numbers 1202, 1203, and 1204 contains circles of three different colors that are intermingled, those colors corresponding to cells assigned the cell types Lymphs, CD4+ T Cells, and CD4+ CD45RA+. This intermingling may interfere with the usefulness of this visualization for some purposes.

While FIG. 12 and each of the display diagrams discussed below show a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

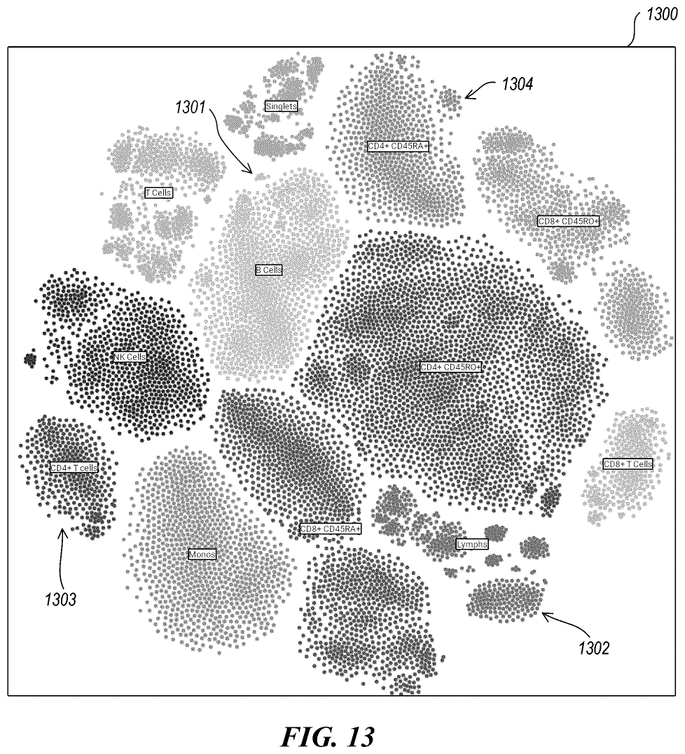

FIG. 13 is a visualization diagram showing a visualization generated by the facility for the example cell sample using an emphasis weight value of 0.25. In the visualization 1300, B Cell's region 1301 is somewhat more tightly clustered than B Cells 1201 shown in FIG. 12. Regions 1302, 1303, and 1304, representing Lymphs, CD4+ T Cells, and CD4+ CD45RA+ cells, respectively, are now distinct, as contrasted with their intermingling at reference numbers 1202, 1203, and 1204 in FIG. 12. Thus, the greater emphasis on cell types resulting from choosing W=0.25 rather than W=0 has helped to be able to see and visually absorb the locations of the cells in a particular cell type, while preserving a proximity between cells of different cell types that nonetheless have similar constituent expression levels.

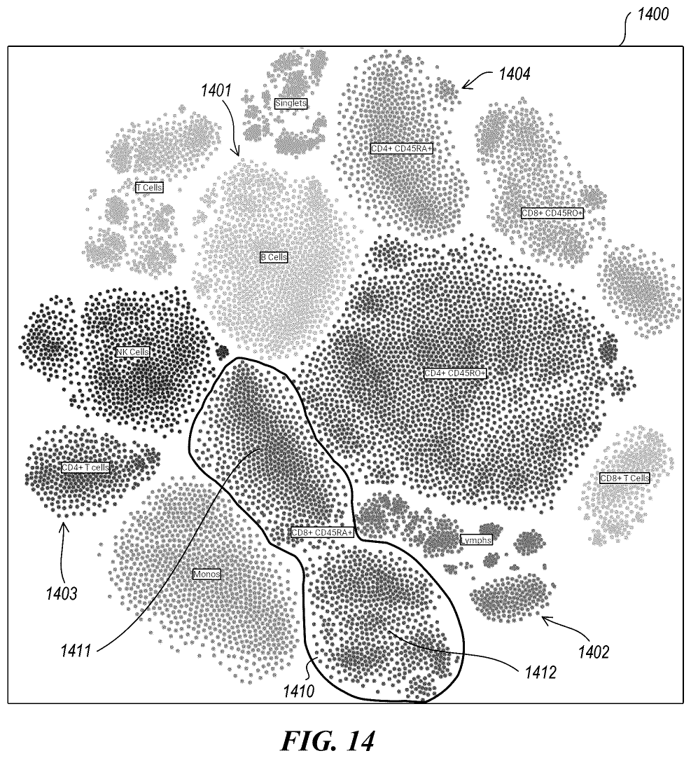

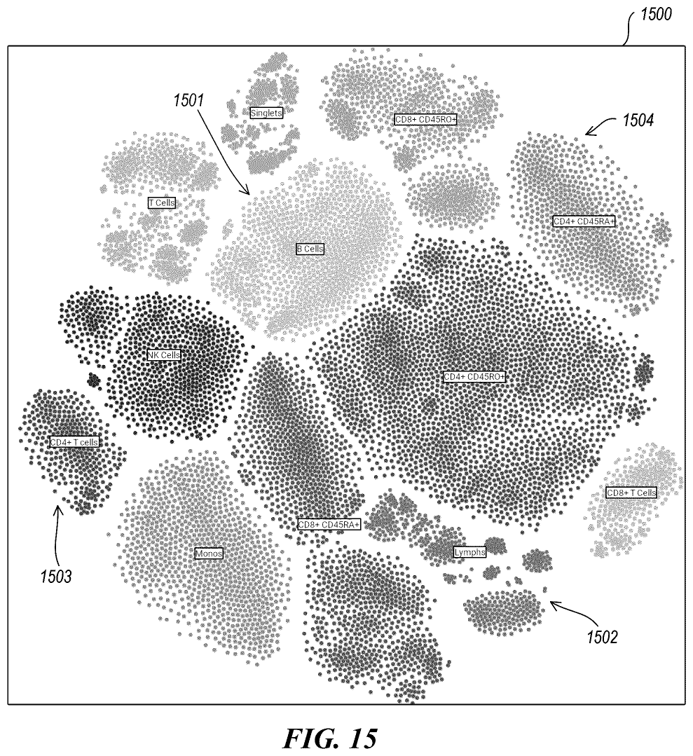

FIG. 14 is a visualization diagram showing a visualization generated by the facility for the example cell sample using an emphasis weight value of 0.5. FIG. 15 is a visualization diagram showing a visualization generated by the facility for the example cell sample using an emphasis weight value of 0.75. As the value of the emphasis weight increases the level of separation between clusters of cells of the same cell types continues to increase.

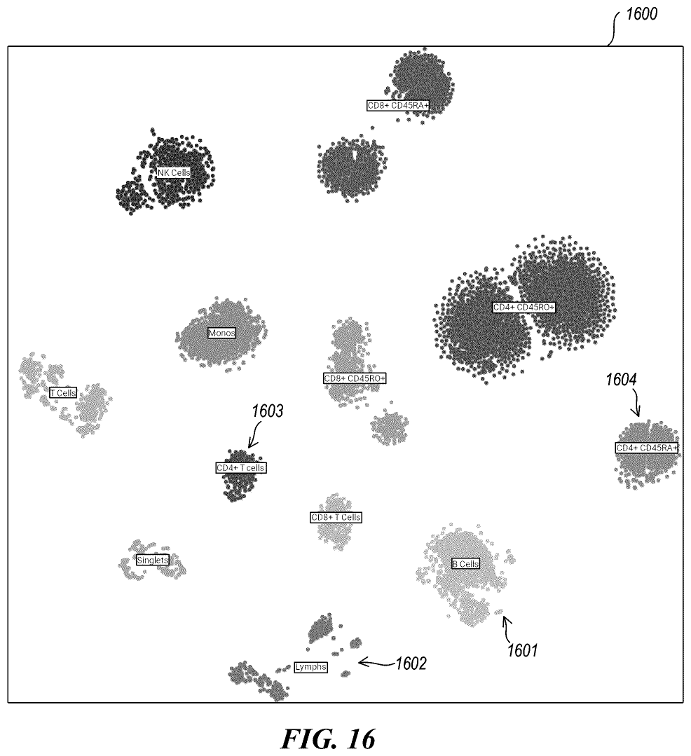

FIG. 16 is a visualization diagram showing a visualization generated by the facility for the example cell sample using an emphasis weight value of 0.99. In the visualization 1600, the cells of individual cell types are very tightly clustered and remote from the other groups, making it difficult to visualize similarities in cellular constituents between groups and cell types.

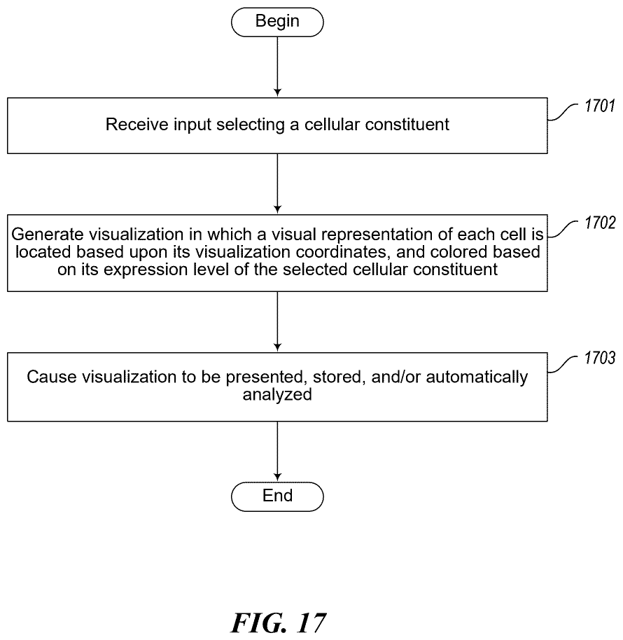

FIG. 17 is a flow diagram showing a process performed by the facility in some embodiments to generate an alternate visualization in which cell visual representation color represents the expression level of a particular constituent. This is useful, for example, when seeking to understand the span of a large group of circles for a particular cell type, and the causes for: the significant distance between certain of them, the differential distance to circles of other cell types, etc. For example, in FIG. 14, a group 1410 of CD8+ CD45RA+ cells spans a large area, and includes two distinct lobes 1411 and 1412. Using this process can help to delineate the relationship of the cells in the two lobes to each other, as well as to cells of other cell types.

In act 1701, the facility receives input selecting a cellular constituent, such as cellular constituent Hu.CD27. In act 1705, the facility generates a visualization in which a visual representation of each cell is located based upon its visualization coordinates and the visual representation of each cell is colored based on its expression level of the cellular constituent selected in act 1701. In act 1703, the facility causes the visualization generated in the act 1702 to be presented, stored, and/or automatically analyzed. After act 1703, this process concludes.

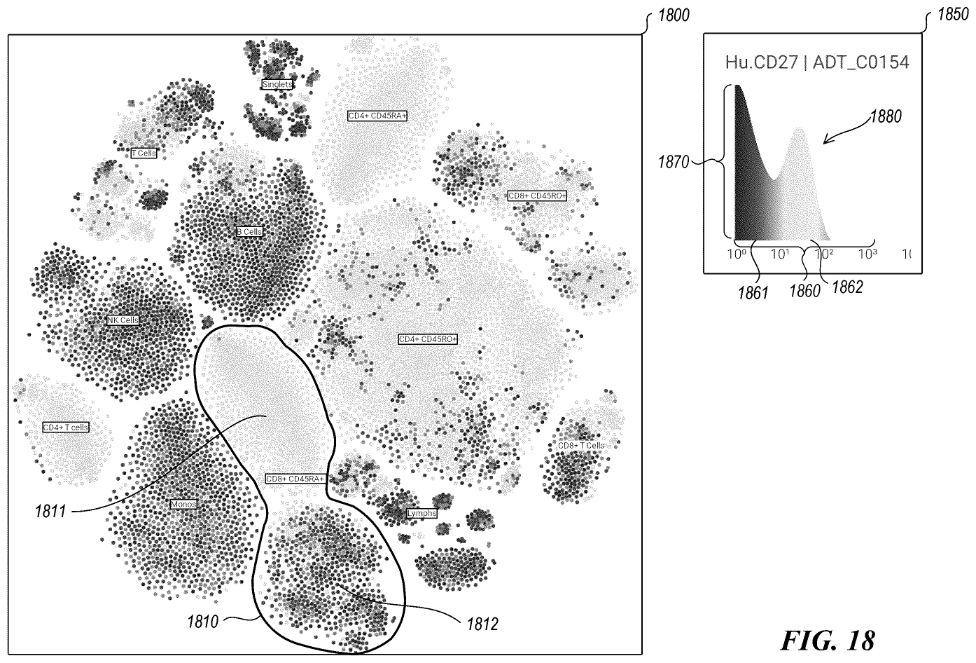

FIG. 18 is a visualization diagram showing a visualization generated in accordance with the process shown in FIG. 17. The visualization 1800 contains the same circles as in visualization 1400 shown in FIG. 14, but they are colored differently in accordance with the process of FIG. 17. In particular, the colors reflect different expression levels of the cellular constituent Hu.CD27. The correspondence between color and expression level is shown in graph 1850 in which curve 1880 shows a frequency 1870 of different expression levels of this constituent among all of the cells of the sample, at each of a number of different expression levels 1860. For example, a blue color is assigned to a lower expression level 1861 of about 3 (between 10⁰ and 10¹) and a yellowish-greenish color at expression level 1962, approximately 85 (i.e., between 10¹ and 10²). By examining the circles in group 1810, it can be seen that those in lobe 1811 have colors corresponding to higher expression level 1962 of Hu.CD27 (i.e., are yellowish-greenish), while the circles in lobe 1812 correspond to lower expression level 1861 (i.e., are bluish).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system performed with respect to a sample of cells, the method comprising:
accessing analysis results indicating a detected expression level for each of a number of cellular constituents of each cell of the sample;
accessing a cell type for each cell of the sample, where the cell type is among a plurality of cell types that is attributed to the cell based on each cell's cellular constituent expression levels;
for each of a plurality of pairs of cell types among the plurality of cell types, accessing a level of similarity between the cell types of each pair;
for each of the plurality of cell types, establishing a first representation of each cell type based on the accessed levels of similarity between each cell type and each of the other cell types of the plurality;
accessing an emphasis weight specifying a degree to which cell type is to be emphasized relative to cell constituent expression levels in determining visualization coordinates for each cell of the sample;
generating a cell matrix comprising a grid of values in which each row represents one of the cells of the sample, in which a first group of the columns correspond to constituent expression levels detected for the cell, and a second group of the columns correspond to the first representation established for the cell's cell type, the values in the first group of the columns being weighted against the values in the second group of the columns in accordance with the accessed emphasis weight; and
performing dimensionality reduction on the rows of the generated cell matrix to obtain visualization coordinates for each cell of the sample.

2. The method of claim 1, further comprising constructing a visualization image containing, for each cell of the sample, a visual indication of the cell that appears in a spatial location specified by the visualization coordinates obtained for the cell.

3. The method of claim 2, further comprising causing the constructed visualization image to be presented.

4. The method of claim 2, further comprising causing the constructed visualization image to be persistently stored.

5. The method of claim 2, further comprising invoking an automatic analysis against the constructed visualization image.

6. The method of claim 1 wherein, in the constructed visualization image, each visual indication of a cell is shown in a color corresponding to the cell's cell type.

7. The method of claim 1 wherein, in the constructed visualization image, each visual indication of a cell is shown in a color corresponding to the expression level indicated for a distinguished cellular constituent in the cell.

8. The method of claim 7, further comprising receiving input specifying the distinguished cellular constituent.

9. The method of claim 1 wherein, for each of the plurality of cell types, establishing a first representation of the cell type comprises:
for each of the plurality of pairs of cell types, accessing a distance in an adjacency graph among the plurality of cell types reflecting a hierarchy established for the plurality of cell types;
for each of the cell types of the plurality of cell types, constructing a second representation of the cell type by concatenating values that are based on the distances accessed for the cell type with respect to all of the cell types of the plurality of cell types;
performing embeddings into an embedding space of the constructed second representations of the cell types of the plurality of cell types to obtain the first representations of the cell types of the plurality of cell types.

10. The method of claim 9 wherein embedding is performed using a process selected from among:
t-distributed Stochastic Neighbor Embedding (t-SNE);
Multi-dimensional scaling (MDS);
Force-Directed Placement;
Kamada algorithm for drawing general undirected graphs; and
Kobourov Spring Embedders and Force-Directed Graph Drawing Algorithms.

11. The method of claim 9, further comprising:
receiving input specifying the hierarchy established for the plurality of cell types;
constructing the adjacency graph in accordance with the hierarchy, in which each of the plurality of cell types is represented by a node, and nodes are connected directly or indirectly by edges; and
for each of the plurality of pairs of cell types, determining the accessed distance by counting the minimum number of edges between the pair of nodes representing the pair of cell types.

12. The method of claim 9, further comprising:
for each of the cell types, determining a frequency of the cell type within the sample; and
determining the values that are based on the distances accessed for the cell type with respect to all of the cell types of the plurality of cell types by weighting the accessed distances in accordance with the determined frequencies.

13. The method of claim 1, further comprising receiving input specifying the accessed emphasis weight.

14. The method of claim 1 wherein the dimensionality reduction is performed using a process selected from among:
t-distributed Stochastic Neighbor Embedding (t-SNE); and
Uniform Manifold Approximation and Projection (UMAP).

15. One or more instances of computer-readable media collectively having contents configured to cause a computing system to perform a method, the method comprising:
accessing analysis results indicating a detected expression level for each of a number of cellular constituents of each cell of the sample;
accessing a cell type for each cell of the sample, where the cell type is among a plurality of cell types that is attributed to the cell based on each cell's cellular constituent expression levels;
for each of a plurality of pairs of cell types among the plurality of cell types, accessing a level of similarity between the cell types of each pair;
for each of the plurality of cell types, establishing a first representation of each cell type based on the accessed levels of similarity between each cell type and each of the other cell types of the plurality;
accessing an emphasis weight specifying a degree to which cell type is to be emphasized relative to cell constituent expression levels in determining visualization coordinates for each cell of the sample;
generating a cell matrix comprising a grid of values in which each row represents one of the cells of the sample, in which a first group of the columns correspond to constituent expression levels detected for the cell, and a second group of the columns correspond to the first representation established for the cell's cell type, the values in the first group of the columns being weighted against the values in the second group of the columns in accordance with the accessed emphasis weight; and
performing dimensionality reduction on the rows of the generated cell matrix to obtain visualization coordinates for each cell of the sample.

16. One or more memories collectively storing a cell matrix data structure, the data structure comprising:
a plurality of first entries, each first entry corresponding to a cell among a plurality of cells comprising a cell sample, each first entry comprising:
a first group of one or more values collectively representing expression levels of cellular constituents detected in the cell to which the first entry corresponds; and
a second group of one or more values collectively comprising a representation of a cell type determined for the cell, assigned such that a distance between the representations of a pair of cell types is representative of a level of dissimilarity between the cell types of the pair,
wherein the second group of one or more values are weighted against the first group of one or more values in accordance with an emphasis weight,
such that the contents of the data structure are usable to create a visualization image for the cell sample comprising, for each cell of the plurality of cells, a visual indication of the cell placed at a spatial location determined based on the contents of the first entry that corresponds to the cell.

17. The one or more memories of claim 16 wherein each first entry of the plurality of first entries further comprises:
coordinates determined for the visual indication of the cell to which the first entry corresponds based on the first and second groups of values of the first entry.

18. The one or more memories of claim 16, the data structure further comprising:
data representing a visualization image for the cell sample, comprising, for each of the plurality of cells of the cell sample, a visual indication of the cell placed at a spatial location determined based on the contents of the first entry that corresponds to the cell, the visual indication being colored in accordance with the cell type determined for the cell.

19. The one or more memories of claim 16, the data structure further comprising:
data representing a visualization image for the cell sample, comprising, for each of the plurality of cells of the cell sample, a visual indication of the cell placed at a spatial location determined based on the contents of the first entry that corresponds to the cell, the visual indication being colored in accordance with an expression level detected in the cell of a distinguished cellular constituent.

20. The one or more memories of claim 16, the data structure further comprising:
a plurality of second entries, each second entry corresponding to one of a plurality of cell types determined for the cells of the plurality of cells of the sample, each second entry comprising:
the representation of the cell type assigned such that the distance between a pair of cell types is representative of a level of dissimilarity between the cell types of the pair.
